(19) United States
(12) Patent Application Publication
(10) Pub. No.: US 2025/0221861 A1
(43) Pub. Date: Jul. 10, 2025

(54) DEVICE FOR SELECTIVE ATTENUATION OF NOISE EMITTED BY DENTISTRY EQUIPMENT

(71) Applicant: SCORPION, ROMAGNAT (FR)

(72) Inventor: STÉPHANE GIGAULT, CHANONAT (FR)

(21) Appl. No.: 18/850,759

(22) PCT Filed: Apr. 7, 2023
(86) PCT No.: PCT/FR2023/050504

§ 371 Date: Sep. 25, 2024

(30) Foreign Application Priority Data
Apr. 27, 2022 (FR) 2203950

(51) Int. Cl. A61F 11/08; A61C 19/00

(52) U.S. Cl. CPC A61F 11/085; A61C 19/00

(57) ABSTRACT

The invention relates to a device (1) for the selective attenuation of noise emitted by dental equipment with a so-called active part (3) whose shapes and dimensions correspond to the shapes and dimensions of an human external auditory canal (4) being and adapted to be removably inserted into the canal (4), a gripping part (5) of the device (1), the active part (3) with at least one communication orifice (71, 81, 91) with the external auditory canal (4) and at least one tubular resonance chamber (7, 8, 9) having an open end (71, 81, 91) and a closed end. The active part (3) is configured as a cylinder with at least two straight, circular-based cylindrical resonance chambers (7, 8, 9) whose longitudinal axes (A7, A8, A9) are parallel to each other and to a longitudinal axis (A3) of the active part (3). At least two resonance chambers have different lengths.

The invention relates to a device for the selective attenuation of noise emitted by dental equipment. Selective means attenuating noise emitted within a given frequency range, while not attenuating noise emitted outside this frequency range.

### DESCRIPTION OF THE RELATED ART

The invention concerns a device intended for use in the medical field, more particularly in the dental field, whether the device is used by a patient and/or by a practitioner. When the practitioner, that is, the dentist, performs certain procedures on a patient, this is done using tools with a moving part, such as a rotating part in the case of a bur, or a vibrating part in the case of a scaling tip. These tools are set in motion by electrical or pneumatic components that generate noise. These vibrating or rotating electric or pneumatic elements emit sounds with a relatively high frequency, generally between 5600 Hz and 6500 Hz. Such noises generally have a sound power of between 50 decibels and 80 decibels. Since the human ear hears sounds emitted in a frequency range from 20 Hz to 20,000 Hz, the above-mentioned frequencies are audible to humans. However, it is generally accepted that the range of frequencies considered pleasant, that is, bearable and not causing hearing discomfort, is between 500 Hz and 4000 Hz, bearing in mind that the frequencies of the human voice range from 75 Hz to around 450 Hz in a child. It is therefore easy to see that devices used in the dental field are potentially a source of hearing discomfort for both patient and dentist, especially at the noise levels encountered.

As a result, both the practitioner and the patient are subjected to noise pollution during the procedure, at frequencies and/or sound levels which, in addition to causing annoyance and discomfort, also limit the dialog the practitioner can have with the patient and/or any other person present during the procedure on the patient. Moreover, while for a patient this noise disturbance is limited in time to the duration of the operation, this is not the case for the practitioner and anyone else working in the dental practice, who are subjected throughout their working day to frequent periods of noise of varying duration. Such a situation can lead to discomfort at work, or even working conditions that are difficult to tolerate, and can have repercussions on the health of people who are sensitive to these sound frequencies.

There is therefore a need for a means of eliminating, or at least reducing, the noise generated by the various dental devices during operations, in a way that is simple but does not affect speech and communication between people, particularly between practitioner and patient. To remedy this, we know of devices such as earmuffs which, in addition to being bulky and complex in construction and therefore relatively expensive, do not provide selective noise attenuation or suppression. Also known from EP-B-3 025 326 is a device for suppressing the noise generated by a dentist's drill. The device is intended for intra-auricular use, that is, allowing easy insertion and removal of the device in the patient's external auditory canal. The device features tubular resonance chambers for selective sound attenuation. These resonance chambers are organized in multiple superimposed layers, each layer having at least three resonance chambers. They are configured as curved spirals around a central pipe uniting them. This gives each layer a rough octopus shape. Such a known solution provides selective noise reduction while remaining easy to insert into an ear canal and therefore easy to use. While such a device provides relatively effective noise reduction in the range of frequencies considered, and is easy to use, enabling communication between patient and practitioner, the structure is nonetheless relatively complex, since a plurality of resonance chambers of relatively small dimensions, that is, of the order of a few millimeters in length, and above all in the shape of a curved spiral, have to be formed. Because of its configuration and construction, such a device is more likely to be reused. What's more, such a device ensures a given level of noise reduction, both in terms of reduced frequencies and reduction volume. U.S. Pat. No. 6,754,359 is an intelligent hearing protection device with wireless communication. This device uses a microphone to capture the sound field in the inner part of the ear canal and, after processing, restore the natural feel of the user's voice when they speak. The sound attenuation is passive, with an earplug provided with a sealing system and active noise control via two microphones and a speaker connected to an electronic unit. U.S. Pat. No. 7,740,104 discloses an earplug comprising multiple chambers with complex geometric shapes and configured as coupled acoustic resonators. Each chamber is provided with an acoustic element having an inertia and resistance coupling the chamber to its neighbor to form multiple resonant chambers. Both documents describe complex devices.

### BRIEF SUMMARY OF THE INVENTION

There is therefore a need for a device that is simpler in structure and therefore less costly to produce than that known in the prior art, which de facto makes it possible to produce a device that is easily adaptable in terms of frequency range and reduction volume, while at the same time being treatable as single-use. Indeed, one of the problems with a reusable device is that when it is intended for patients, it must be cleaned and disinfected between each patient, which generates constraints in terms of time, mobilization of personnel and materials, as well as additional costs for the dental practice.

The object of the invention is to propose a device for optimizing selective noise reduction in a dental practice, while allowing dialog between people, of simple construction and easy use, requiring no maintenance, while being adaptable in terms of reduced frequencies and reduction volume.

To this end, the object of the invention is a device for selectively attenuating noise emitted by dental equipment at frequencies between 5600 Hz and 6500 Hz without attenuating the frequencies between 75 Hz and 450 Hz corresponding to the human voice, said device comprising a so-called active part whose shapes and dimensions correspond to the shapes and dimensions of an external auditory canal of a human being and adapted to be removably inserted into the external auditory canal, a gripping part of the device, the active part comprising at least one orifice for communication with the external auditory canal and at least one tubular resonance chamber having an open end and a closed end, characterized in that the active part is configured as a cylindrical bundle of tubular resonance chambers comprising at least two straight, circular-based cylindrical resonance chambers whose longitudinal axes are parallel to each other and to a longitudinal axis of the active part, and in that at least two resonance chambers have different lengths, and in that the internal diameter of the resonance chambers is between 2 mm and 5 mm.

The invention thus provides a simple, low-cost means of selectively attenuating the noise emitted by dental equipment. The number of resonance chambers and their dimensions can thus be varied to provide a suitable reduction in terms of attenuated volume and attenuated frequencies. This is because the attenuation capacity of a resonance chamber varies according to its geometric characteristics. In particular, a chamber with a smaller volume will reduce noise less than a chamber attenuating the same frequency but with a larger volume.

According to advantageous but optional aspects of the invention, such a device may comprise one or more of the following features:

The number of circular cylindrical resonance chambers is three.

All resonance chambers have the same internal diameter.

At least two resonance chambers have different internal diameters.

At least one of the resonance chambers has a diameter of around 3.3 mm.

The invention also relates to a kit comprising at least two devices complying with one of the preceding features, each device having a different attenuation volume and/or frequency range from the other device.

### BRIEF DESCRIPTION OF THE SEVERAL VIEW OF THE DRAWING

The invention will be better understood and other advantages thereof will become more clearly apparent on reading the following description, given solely by way of non-limiting example and with reference to the accompanying drawings, wherein:

FIG. 1 is a simplified perspective view of a human ear equipped with a device according to one embodiment of the invention,

FIG. 2 is a perspective view, on a larger scale and from a different angle, of the device shown in FIG. 1, and

FIG. 3 is a cross-section, at a different scale, along section plan III-III.

### DETAILED DESCRIPTION OF THE INVENTION

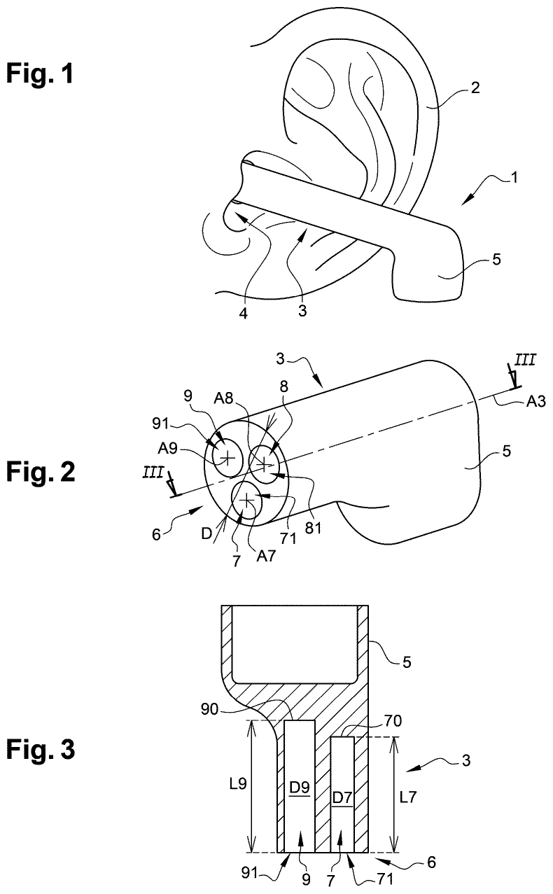

FIG. 1 is an illustration of one embodiment of the invention. Here, a device 1 is shown in place in an ear 2 of a human being, that is, an adult or child.

As can be seen from the various figures, the device 1 comprises a so-called active part, hereinafter referenced as 3, which is inserted at least partially into the external auditory canal 4 of the human ear 2. For the record, the external auditory canal is the part of the ear in contact with the outside world. It ends with the eardrum, which both isolates the inner ear from direct contact with the outside world and ensures sound transmission by vibration, the eardrum being a membrane. When a foreign body is inserted into the external auditory canal 4, this foreign body only penetrates a few millimeters, as the geometric configuration of the canal 4 prevents it from reaching the eardrum, at the risk of discomfort or pain.

To facilitate insertion and removal of this active part 3 in the auditory canal 4, the active part 3 is extended by a gripping part 5. Here, this gripping part 5 is configured as a larger zone, relatively flattened and arranged at an angle to the active part 3. As can be seen from FIG. 3, the gripping part 5 is hollow in order to lighten the weight of the device and limit manufacturing costs. Alternatively, the gripping part is solid or configured as a flat tab or ring.

The active part 3 is generally shaped like a cylinder with a circular base, so that it can easily adapt to the configuration of an external auditory canal 4 in its accessible portion, which is de facto the portion formed in the vicinity of the outlet of the auditory canal 4 at the ear 2. It is easy to understand that the material making up the active part 3, at least on its outer surface which is in contact with the inside of the auditory canal 4 when part 3 is in position in the auditory canal 4, is a lightweight material which is biologically compatible and therefore non-allergenic for a human being. The active part 3 inserted in the duct 4 does not seal the duct 4 and therefore allows sound transmission between the outside world and the eardrum. To achieve this, the material of active part 3 is a rigid material with a relatively high Young's modulus, so that it resonates at maximum amplitude, without damping. In a preferred embodiment, the active part 3 and the gripping part 5 are single-piece and made of the same material. Alternatively, the material of each part is different. The device 1 is produced by molding, injection, machining or any other technique known per se.

In order to produce a device 1 which, while easy to handle, remains relatively unobtrusive when in place in the auditory canal 4, the overall length of the device 1, that is, comprising the active part 3 and the gripping part 5, is between 20 mm and 30 mm, bearing in mind that the overall external diameter D of the active part 3 is at most around 9 mm, which enables the material making up this active part 3 to adapt to the internal diameter of most auditory canals 4, at least in adults. For use by children, the maximum diameter of the active part 3 must not exceed 8 mm.

FIG. 1 shows a device 1 in place in the ear canal 4 of one ear 2, on the understanding that the other ear is equipped with the same device, otherwise there will be no noise attenuation for the person equipped with a single device, unless that person has lost the use of the ear not equipped with the device of the invention. Alternatively, each device 1 is provided with a visual marking to identify whether it is intended to be used for the right or left ear. According to another embodiment, the two devices 1 are connected together by a cord to prevent any loss, for example when they accidentally leave the ear canal 4 in the event of incorrect insertion.

FIG. 2 shows the device shown in the figure from the free end 6 first inserted into the auditory canal 4, that is, from the end opposite the end of the active part 3 connected to the gripping part 5. The active part 3 comprises at least two resonance chambers. Advantageously, as shown in FIG. 2, the active part 3 has three resonance chambers, referenced 7, 8 and 9. These resonance chambers 7 to 9 open out at one end onto the edge of end 6. They are formed by hollow cylinders with a circular base, straight lines and a closed end. FIG. 3 shows the cross-section of two chambers, chambers 7 and 9, and shows the flat-bottomed closed ends 70 and 90 and the open ends 71 to 91 of these chambers, it being understood that chamber 8 also has a flat-bottomed closed end and an open end. These chambers 7 to 9 are arranged with their respective longitudinal axes A7, A8 and A9 parallel to each other and to a longitudinal axis A3 of the active part 3. In other words, the active part 3 can be considered as a bundle of straight tubular resonance chambers 7 to 9 united in a common cylindrical structure. At least two of these resonance chambers 7 to 9 have different lengths. Here, FIG. 3 shows the different lengths L7 and L9 of chambers 7 and 9. In another embodiment, all the resonance chambers have different lengths. Internal diameters D7 and D9 may be identical or, as shown in FIG. 3, different for at least two of the chambers. Alternatively, the diameters of the chambers are all different, and the lengths of some of the chambers may be equal. The difference between the diameters of each chamber, taking into account the maximum diameter D of part 3 and the number of chambers, is between 0.1 mm and 0.8 mm. Here, the diameters of chambers 7 to 9 in FIG. 2 are shown as equal for ease of reading. In another embodiment, all the chambers have not only different lengths but also different diameters.

Because of the maximum diameter D required of the active part 3, at least one of these cylindrical resonance chambers 7 to 9 has an internal diameter of around 3.3 mm, while the other chambers can have diameters of between 2 mm and 5 mm, that is, possibly also equal to 3.3 mm.

FIG. 3 shows the case where the lengths L7, L9 and diameters D7, D9 of at least two of the resonance chambers 7 and 9 are not identical. It is a well-known fact that the sound attenuation achieved by a resonance chamber is a function of its volume. In the case of cylindrical resonance chambers with circular and rectilinear bases, such as those covered by the invention, the attenuated frequency varies, with equal diameters, as a function of the length of the resonance chamber, that is, as a function of the length of the cylinder within the active part 3, between the opening and the closed end of the cylinder. The shorter the length of a chamber, given the same internal diameter, the greater the attenuation at higher frequencies. Thus, for an internal diameter of 3.3 mm, attenuation occurs at 6010 Hz for a length of 13.2 mm, at 6150 Hz for a length of 12.9 mm and at 6380 Hz for a length of 12.4 mm.

The quality of attenuation, that is, the volume of noise reduction, is expressed in decibels and depends, in this case, on the diameter of the resonance chamber in question. As such, given equal lengths and equal attenuated frequencies, the greater the internal diameter of a resonance chamber, the greater its attenuation. By way of example, for the same frequency and length, the reduction varies by five decibels between a chamber with a diameter of 4 mm and one with a diameter of 5 mm.

Owing to the invention, resonance chambers 7 to 9 of different lengths can be used to attenuate noise emitted by dental equipment over a relatively wide frequency range, generally between 5600 Hz and 6500 Hz. Selective noise reduction at these frequencies means that sounds emitted at lower frequencies, particularly those between 75 Hz and 450 Hz, which correspond to the frequencies generally encountered in the human voice, are not attenuated, at least not significantly. Thus, voice exchanges between people present are not, or only slightly, affected by the wearing of the device 1 by those present.

With a device 1 comprising three chambers with identical diameters of around 3.3 mm and different lengths varying between 12.4 mm and 13.2 mm, it has been found that sound intensity can be reduced by at least 40 dB for frequencies between 5900 Hz and 6500 Hz and by at least 30 dB for frequencies between 5600 Hz and 5900 Hz.

According to another embodiment, the invention also comprises a kit of at least two devices 1. Advantageously, the kit comprises more than two devices 1. These devices can cover a wider range of frequencies and/or with greater reductions. In other words, in a kit comprising at least two devices 1, these have characteristics in terms of lengths and/or diameters that are different in their respective resonance chambers. Alternatively, such kits can be produced with devices having, in addition to variable lengths and/or diameters, a different number of chambers and/or a different external diameter of the active part, in order to offer greater adaptability in terms of sound attenuation, for example for adults and children. In order to differentiate between the various devices, depending on their intended use (adult or child) and/or their attenuation characteristics in terms of volume and/or frequency, visual markings are used. It is also conceivable that, in the same kit, several copies of the same device are present, in order to be able to equip multiple people with a device that is similar in terms of performance.

The invention provides dental practices with a set of devices that are easy to insert into an ear canal, whether by the patient or the practitioner, and which attenuate noise without affecting conversations between those present.

The simple structure in a relatively inexpensive material enables hearing protection devices to be produced using techniques known per se, which are not intended to be reused and can therefore be considered single-use consumables. Alternatively, of course, it is possible to have devices that allow cleaning and disinfection for reuse of the device.

1. A device (1) for selectively attenuating noise emitted by dental equipment at frequencies between 5600 Hz and 6500 Hz without attenuating the frequencies between 75 Hz and 450 Hz corresponding to the human voice, said device comprising a so-called active part (3) whose shapes and dimensions correspond to the shapes and dimensions of an external auditory canal (4) of a human being and adapted to be removably inserted into the external auditory canal (4), a gripping part (5) of the device (1), the active part (3) comprising at least one orifice (71, 81, 91) for communication with the external auditory canal (4) and at least one tubular resonance chamber (7, 8, 9) having an open end (71, 81, 91) and a closed end (70, 90), characterized in that the active part (3) is configured as a cylindrical bundle of tubular resonance chambers comprising at least two straight, circular-based cylindrical resonance chambers (7, 8, 9) whose longitudinal axes (A7, A8, A9) are parallel to each other and to a longitudinal axis (A3) of the active part (3), and in that at least two resonance chambers (7, 9) have different lengths (L7, L9), and in that the internal diameter of the resonance chambers (7, 8, 9) is between 2 mm and 5 mm.

2. The device according to claim 1, characterized in that the number of circular-based cylindrical resonance chambers (7, 8, 9) is three.

3. The device according to claim 1, characterized in that all the resonance chambers have the same internal diameter.

4. The device according to claim 1, characterized in that at least two resonance chambers (7, 9) have a different internal diameter (D7, D9).

5. The device according to claim 1, characterized in that at least one of the resonance chambers (7, 8, 9) has a diameter of around 3.3 mm.

6. A kit comprising at least two devices (1) in accordance with claim 1, each device (1) having a different attenuation volume and/or frequency range from the other device (1).
